(19) United States
(12) Patent Application Publication
(10) Pub. No.: US 2004/0161433 A1
(43) Pub. Date: Aug. 19, 2004

(54) Method for in vitro culture of lymphocytes and composition for use in immune therapy

(76) Inventors: Keisuke Teshigawara, Kyoto-shi (JP); Yuji Ohkubo, Kyoto-shi (JP)

Correspondence Address: BIRCH STEWART KOLASCH & BIRCH, PO BOX 747, FALLS CHURCH, VA, 22040-0747, US

(21) Appl. No.: 10/646,743

(22) Filed: Aug. 25, 2003

Related U.S. Application Data
(63) Continuation-in-part of application No. 09/868,779, filed on Aug. 20, 2001, now abandoned.
Application No. 09/868,779 is a 371 of international application No. PCT/JP00/07385, filed on Oct. 23, 2000.

(30) Foreign Application Priority Data
Oct. 21, 1999 (JP) 11-300122

(51) Int. Cl. A61K 39/00

(52) U.S. Cl. 424/277.1; 424/93.7

(57) ABSTRACT

The method for in vitro culture of lymphocytes involves incubating lymphocytes and cells with a particular gene expressed in a particular cancer cell line or cells, which are deficient or lowered in expression of a class 1 antigen and the particular gene has already been expressed, to amplify mainly NK cells or non-MHC-bound or MHC-bound killer T cells and then to amplify killer T cells specific to an antigen to a cancer. The in vitro culture of the lymphocytes can produce a group of lymphocytes composed mainly of killer cells.

The group of the lymphocytes so produced can be used for immune therapy effective even for patients with cancer in the terminal stage to whom conventional cancer treatment and cancer curing agents.

The present application is a Continuation-in-Part (CIP) of application Ser. No. 09/868,779 filed on Aug. 20, 2001 (now abandoned), which was the national phase under 35 U.S.C. § 371 of PCT International Application Number PCT/JP00/07835 which has an International filing date of Oct. 23, 2000, and which designated the United States of America and was not published in English, both applications of which are hereby incorporated by reference.

### TECHNICAL FIELD

The present invention relates to a novel method of in vitro culture for the multiplication of lymphocytes and a novel composition for use in immune therapy by using amplified lymphocytes. More particularly, the present invention relates particularly to a composition for use in immune therapy, which can realize a remarkably useful cancer therapy that can be effective even for patients with cancer ineffective by conventional cancer therapy.

### TECHNICAL BACKGROUND

In order to culture solely a lymphocyte group consisting of NK cells for a long period of time under in vitro circumstances, there have hitherto been used mainly two methods. (1) One method involves using autologous B cells for the maintenance of culturing NK cells, the B cells being modified by EB virus and then irradiated with radiation to repress the multiplication of NK cells. This known method can maintain the culture of NK cells, but it requires purification of NK cells and establishment of autologous B cell line. (2) The other method involves culturing NK cells from lymphocytes with IL2 without purification. The resulting NK cells have been used for LAK therapy. This method, however, can multiply the NK cells by several times only and the effects achieved by this therapy are limited.

Further, the method (1) suffers from the problems that it has to use EB virus, which is known as a virus involved with oncogenesis, and that the therapy using a lymphocyte group consisting of such NK cells may cause serious side effects, etc. Moreover, the modified B cells vary to a great extent in ability concerning therapy effects so that it is very difficult to achieve stable culture. In addition, this method requires purification of NK cells so that a loss of cells is caused and a great amount of labor is required.

On the other hand, the method (2) has the defects that it can multiply NK cells by several times only so that the ability of multiplying NK cells is low and, if the culture would have been carried out for a long period of time, T cells having no killer activity may be caused to multiply selectively, therefore, the therapy effects are limited.

With the above background taken into account, the present inventors have conducted extensive review and studies on a method for in vitro culturing lymphocytes which can stably produce a lymphocyte group effective for cancer therapy yet which does not use virus such as EB virus, etc. involved with oncogenesis, and which does not require purification of NK cells. As a result, the present inventors have found a novel method for in vitro culturing lymphocytes by using an approach that is thoroughly different from conventional methods for culturing NK cells and that can produce such a lymphocyte group stably. More specifically, it has been found that this method can multiply a lymphocyte group consisting of activated NK cells and CD4-positive T cells in a safe and stable manner and that such a lymphocyte group can be used as a source of conventional adoptive immune therapy. It is further found that the NK cells grown by this method have the activity of damaging cancer cells higher than those grown by conventional methods.

On the basis of the studies performed, the present inventors have previously proposed an in vitro culture method for multiplying a lymphocyte group having a high killer activity concerning a cancer cell-damaging activity by culturing a combination of class I-negative (or the expression of class I is low) cancer cells with B7 gene expressed therein and lymphocytes derived from peripheral blood with an immunomodulator at various rates (Japanese Patent Application No. 59,336/1999; Laid-open No.). It now has been found that this in vitro culture method still has the points to be improved that a lymphocyte group having a high killer activity can be multiplied by several times, but that killer cells selectively derived from such a lymphocyte group so as to be adapted to individual patients cannot be multiplied to more than 10 times.

### DISCLOSURE OF THE INVENTION

With the above situation taken into account, the present inventors have continued extensive review and studies to find a method for in vitro culturing lymphocytes which can selectively derive killer cells having a killer activity so as to adapt to each individual and multiply such killer cells. As a result, the present inventors have found a method for in vitro culturing lymphocytes which can multiply NK cells, or non-MHC-bound or MHC-bound killer cells in combination with killer T cells specific to cancer antigen. It was found that this in vitro culture method can selectively derive and multiply killer cells from the lymphocytes having a killer activity so as to adapt to individual patients without purifying specific precursor cells. It was further found that the killer cells cultured and multiplied by this in vitro culture method can be applied as a source of an immune therapy even for patients with cancer for whom conventional cancer therapy was ineffective, so that this method can realize a remarkably effective cancer therapy. The present invention has been completed based on these findings.

Therefore, the present invention has the object to provide a method for the in vitro culture of lymphocytes that can selectively derive killer cells from lymphocytes having a killer activity and multiply such killer cells. The present invention has another object to provide a composition for use in immune therapy which comprises the killer cells cultured and multiplied by the in vitro culture method and which can be applied as a source of immune therapy.

In order to achieve the above objects, the present invention provides a method for the in vitro culture of lymphocytes, which comprises culturing lymphocytes and a cancer cell which expresses an immunoglobulin superfamily gene, thereby activating the lymphocytes, wherein the cancer cell is deficient or decreased in the expression of a class I antigen, and wherein the immunoglobulin superfamily gene encodes a cell adhesion molecule.

The present invention also provides a method for the in vitro culture of lymphocytes, which comprises culturing lymphocytes and a cell which expresses an immunoglobulin superfamily gene, such as B7 gene, CD40, LFA-1, or a combination thereof, to multiply mainly NK cells, or non-MHC-bound or MHC-bound killer T-cells; or multiplying killer T cells specific to a cancer antigen together with the NK cells or the non-MHC-bound or MHC-bound killer T cells.

In a preferred embodiment of the present invention, there is provided an in vitro culture method for growing lymphocytes which involves using cancer cells, as the particular cancer cells, which are deficient or low in the expression of a class I antigen. Further, in a preferred embodiment, the present invention provides a method for in vitro culture of lymphocytes, which involves using the expression gene consisting of B7 gene or a mixture of B7 gene with a cancer antigen gene or gene(s) of cell adhesion molecules (such as CD40 and/or LFA-1). In a more preferred embodiment, the present invention provides a method for the in vitro culture of lymphocytes, which involves using lymphocytes immediately after the separation from peripheral blood or lymphocytes activated with an immunomodulator that can facilitate damaging cancer cells.

In addition to the culture method for the in vitro growth of the lymphocytes, the present invention in another aspect provides a composition for use in immune therapy, which comprises lymphocytes obtained by amplifying NK cells or killer T cells produced by the in vitro culture method according to the one aspect of the present invention. The lymphocytes that are activated are preferably NK cells. The activated lymphocytes, such as activated NK cells, may be administered to a patient in need thereof for circulation in that patient.

The present invention also provides a method for immunostimulation in a cancer patient or for treating cancer in a patient comprising isolating lymphocytes from the patient, incubating the lymphocytes and a cancer cell which expresses an immunoglobulin superfamily gene, thereby activating the lymphocytes, and administering the activated lymphocytes to the same cancer patient, wherein the cancer cell is deficient or decreased in the expression of a class I antigen, and wherein the immunoglobulin superfamily gene encodes a cell adhesion molecule. In this process of immunostimulation or treatment, the isolation, incubation, and administration steps may be repeated as often as necessary, such as three times or more.

The present invention also provides a method for inducing the proliferation of activated NK cells, comprising isolating lymphocytes from a subject, incubating the lymphocytes and a cancer cell which expresses an immunoglobulin superfamily gene, thereby activating the NK cells, wherein the cancer cell is deficient or decreased in the expression of a class I antigen, and wherein the immunoglobulin superfamily gene encodes a cell adhesion molecule. This method is useful for a healthy subject, who can reserve activated NK cells for possible later-developed cancer diseases.

Other objects, features and advantages will become apparent in the course of the following description.

### BRIEF DESCRIPTION OF THE ACCOMPANYING DRAWINGS

FIG. 1 shows the relationship of NK cells with killer T cells specific to a cancer antigen.

FIG. 2 shows activated NK cells assayed for cell surface markers by FACScan flowcytometry using FITC-labeled anti-CD3 antibody and PE-labeled anti-CD56 antibody.

FIG. 3 shows the amount of cultured cells obtained from patients' blood samples following administration of activated NK cells to the patients.

FIG. 4A shows a photograph and diagram of the morphology of lymphocytes including non-activated NK cells.

FIG. 4B shows a photograph and diagram of the morphology of lymphocytes and activated NK cells.

FIG. 5 shows photographs depicting the change in morphology of NK cells due to stimulation with K562 cells that express B7 gene.

FIG. 6 shows the cytotoxicity % of target cells incubated with non-activated NK cells (NK), T cells from LAK (LAK), and activated NK cells (ANK).

FIG. 7 shows the cytotoxicity % of K562 cells that express B7 gene incubated with non-activated NK cells (NK), T cells from LAK (LAK), and activated NK cells (ANK).

FIG. 8 shows lymphocytes assayed for cell surface markers by FACScan flowcytometry using anti-CD3 antibody and anti-interferon-γ antibody.

### BEST MODES FOR CARRYING OUT THE INVENTION

The method for in vitro culturing lymphocytes according to the present invention involves amplifying mainly non-MHC-bound or MHC-bound killer T cells by culturing lymphocytes in admixture with a cancer cell in which a particular gene is caused to be expressed therein or a cell in which such a particular gene has been expressed therein, such a particular gene including, for example, B7 gene, CD40, LFA-1, or a combination thereof, such as a B7 gene with a cancer antigen gene, and further amplifying killer T cells specific to a cancer antigen.

The lymphocytes to be used as an effecter for the present invention may be collected from patients with cancer (malignant lymphoma, liver cancer, pancreas liver, large intestine cancer, etc.) through leukophoresis or intravenous puncture. The blood collected directly from patients is usually processed by conventional methods to separate lymphocytes and adjusted with RPMI or the like to a predetermined concentration, e.g., 2×10⁶ cells per milliliter. The measurement of the concentration of cells may be conducted with a hemocytometer. A suspension of the lymphocytes with their concentration adjusted to such a predetermined level is used as a lymphocyte stock suspension for use in the in vitro lymphocyte culture. The steps for procedures are similar to conventional ones. The procedures for the method of the present invention may be carried out by preparing a culture medium by diluting the lymphocyte stock suspension to a predetermined optimal concentration.

In accordance with the present invention, as the cells to be used for the in vitro culture of lymphocytes for the expression of the particular gene, there may be used a cancer cell line that is deficient or decreased in the expression of a class 1 antigen. Such a cancer cell line may include, for example, K 562 cell derived from human chronic myelocytic leukemia and Daudi cell derived from lymphoma.

Further, the cancer antigen gene may be collected from cancer cells of a patient with cancer by means of conventional methods such as a stab suction method and identified by means of conventional methods such as PCR method. For instance, the cancer antigen gene may be identified by extracting RNA from the collected cancer cells by means of acid-guanidium thiocyanate/phenol-chloroform extraction method (AGPC method) and detecting the expression of the gene by means of PCR method or DNA chips, etc. using cDNA obtained by RT (Reverse Transcription) reaction as a template.

As the method for the introduction of the particular gene into the cancer cells, there may be used conventional introduction methods which may include, for example, microinjection method, phosphate calcium-DNA co-precipitation method, DEAE-dextran method, liposome method, particle gun method, electroporation method, gene introduction method using a microorganism such as retro virus, adenovirus, herpes virus, protoplast fusion or cell fusion method, and so on. For the method for the expression of the gene introduced into the target cancer cell line, such conventional expression methods can be used. For instance, an expression gene may be bound to an expression vector with an gene resistant to an toxic agent such as neomycin or hygromycin and introduced into the target cells by means of conventional methods such as electroporation method or the like. After the gene has been introduced, the agent such as neomycin, hygromycin or the like is added to kill the cells other than the cells with the target gene introduced therein and to collect only the cells with the target gene expressed therein. The collected cells with the target gene expressed therein are then incubated and amplified by using conventional methods such as limiting dilution-culture method or the like and the amplified cells are used for in vitro culturing lymphocytes. A plurality of genes can also be expressed simultaneously by means of the like method. In order to use plural expression genes, accordingly, it is preferred to use cells in which the plural expression genes are expressed simultaneously. Such gene-expression cells are used as feeder cells.

The method of the in vitro culture of the lymphocytes in accordance with the present invention may be carried out by diluting a lymphocyte culture medium prepared to an optimal concentration with a predetermined concentration of the lymphocyte culture stock suspension, adding the mitomycin-treated or irradiated feeder cells at a predetermined rate to the culture medium and incubating the lymphocytes in the culture medium for a predetermined period of time. This culture does not require special operations and can be done in accordance with conventional procedures. More specifically, the feeder cells are diluted to 2×10⁴ cells per milliliter with a lymphocyte culture adjustment liquid prepared by diluting the lymphocyte culture stock suspension and cultured for a week or longer in a CO₂ incubator. This culture allows the amplification of mainly NK cells or non-MHC-bound or MHC-bound killer T cells as well as killer T cells specific to the cancer antigen. This allows the amplification of killer cells having the activity to damage the cancer cells to approximately several ten times to thousand times or more by co-culturing the cancer cell line to be added at an appropriate frequency. After culturing, the culture liquid is washed with PBS to purify amplified killer cells. The killer cells so cultured and amplified are then returned to the patient, from which the lymphocytes were gathered, by administering them through a dripping pack or the like.

As the lymphocytes, there may be used lymphocytes separated from peripheral blood just after drawing and lymphocytes activated with an immunomodulator that can become likely to damage the cancer cells. As the immunomodulators, there may be mentioned, for example, various cytokines, various biologically response modifiers, various herbaceous materials, nutritious materials contained in various food, or trace metals that may give an influence on an intercellular machinery. These immunomodulators may be used singly or in combination of two or more. Such various cytokines to be used for the present invention may include, for example, IL-1 to IL-18, inclusive, TNF-α, TNF-β, INF-α, INF-β, INF-γ, G-CSF, M-CSF, GM-CSF and so on. Further, such various biological response modifiers may include, for example, OK 432 (Picibanil™, Chugai Pharmaceuticals, Co., Ltd., Japan), sizofiran (Soniflan™, Kaken Pharmaceutical, Co., Ltd.), urinastatin (Miraclid™, Mochida Pharmaceuticals, Co., Ltd.), lentinan, Maruyama vaccine and so on. Moreover, the immunomodulators including such various herbaceous materials, nutritious materials and trace metals are illustrated specifically in Japanese Patent Application Laid-open No. 11-300,122 which is incorporated by reference as part of this description. The immunomodulators may generally be used in the form of an extract liquid or, as needed, in the form of concentrated extract liquid, obtained by drying or pulverizing them by conventional methods that do not damage their components, and extracting them with water or other solvents. Such extract liquid is then admixed with a culture liquid for culturing the lymphocytes.

A detailed description will be given regarding the in vitro culture method for lymphocytes according to the present invention by way of working examples. It is to be noted herein, however, that the present invention is not limited in any respect to those working examples. Further, in the following specification, the B7 gene is described as an expression gene, but it should be understood that the present invention is not intended at all to be limited to the B7 gene.

### WORKING EXAMPLES

### Example I

Procedures of Incubating Cancer Cells with Lymphocytes with the B7 Gene Expressed Therein:

The B7 gene was incorporated into an expression vector having a neomycin-resistant gene, and the expression vector was introduced into K562 cells by means of electroporation method in order to allow the B7 gene to be expressed therein. The K562 cells with the B7 gene expressed therein and the human lymphocytes were cultured in a medium (Hi-Medium™: NIPRO) containing IL-2 to which human serum was added, and they were incubated under 5% CO₂ at 37° C. Although the amount of the culture medium may vary with the amount of the lymphocytes, the human lymphocytes were adjusted to 1−5×10⁶ cells per milliliter in this example. And the K562 cells with the B7 gene expressed therein were added at the rate of 1/100 to 1/500. Further, the B7-expression cells were treated with mitomycin or by irradiation so as to cause no amplification. The B7-expression cells were cloned by the limiting dilution-culture method and the cells having high expression were selected by means of flow rate meter.

Procedures of Separating Lymphocytes as an Effector:

The blood drawn from a patient was poured in a centrifugal tube and diluted to twice with a phosphate buffered solution (PBS), followed by transferring the blood into plural lympho-prep tubes for use in blood separation and centrifuged at 1,500-2,000 rpm at ambient temperature for 15 to 20 minutes. The resulting lymphocyte layer (a layer so-called as buffy coat) was gathered and transferred into a new centrifugal tube and washed with about 30-40 ml per tube of PBS or RPMI 1640, followed by centrifugation at 1,500 rpm for 10 minutes and removal of the resulting supernatant. This operations were repeated twice and a human serum-added medium (Hi-Medium™) was added thereto to adjust the lymphocytes to a predetermined concentration, e.g., 4×10⁶ cells per milliliter. The measurement of the lymphocyte concentration was conducted with a blood count plate and the number of lymphocytes was counted as accurately as possible. The resulting liquid was used as a lymphocyte culture stock liquid.

The lymphocyte culture stock liquid was incubated in substantially the same manner as the above incubation. For example, the culture was carried out by pouring 5 ml of the lymphocyte culture stock liquid into each flask to adjust the number of the feeder cells to 4×10⁴ cells per milliliter, incubating the flask in a CO₂ incubator for one week according to conventional method, washing the lymphocyte culture medium with physiologically saline, and selecting the lymphocytes followed by purification.

The lymphocytes purified in the above manner are then subjected to investigation according to the methods as proposed in our copending Japanese Patent Application Nos. 9-342,675 and 11-174,053. The results of investigation are shown in Table 1 below and reveal that the ability of the lymphocytes for the damaging of the cancer cells was increased by approximately 9 times.

Now, a brief description will be given regarding the method of investigation. A lymphocyte culture medium was prepared by separating the lymphocytes separated from peripheral blood and adding the lymphocytes to a culture medium, followed by incubating the culture medium overnight in an incubator. The lymphocytes so incubated are used as an effecter and the magnitude of Europium radiated from the cells is measured by using K562 cells labeled with Europium as a target by means of fluorophotometer. The results are shown in Table 1 below.

TABLE 1
Comparison of the immune ability of lymphocytes at
the time of gathering blood (A) with the immune ability
of lymphocytes after the application of this invention (B)

| | |
|---|---|
| IMMUNE | RATIO |

| ABILITY (%) | 40:1 | 20:1 | 10:1 | 5:1 |
|---|---|---|---|---|
| (A) | 5.5 | 3.2 | 1.5 | 1.3 |
| (B) | 96.3 | 80.5 | 70.5 | 55.8 |

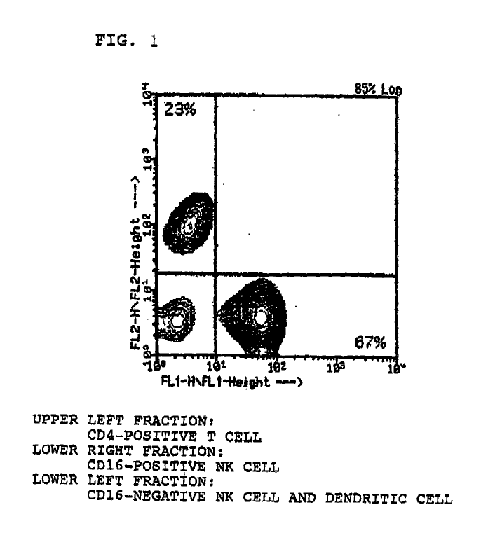

As is apparent from Table 1 above, the lymphocytes prepared by the culture method according to the present invention are found to be a group of lymphocytes, as is shown in FIG. 1, which consist mainly of killer cells having a high activity of damaging the cancer cells. The results as shown in FIG. 1 are those obtained by the measurement with a FACScan flowcytometry (Beckton-Dickinson). From these results, it is indicated that the group of the resulting lymphocytes is composed mainly of NK cells and killer T cells specific to cancer cells.

### Example II

Preparation of Activated NK Cells

(1) Preparation of K562 Cells that Express B7 Gene

The B7 gene was synthesized by RT-PCR using the primer as prepared based on the sequence of a DataBase of The European Molecular Biology Laboratory (EMBL), ENSEEMBL: ENSG00000121594. The B7 gene was incorporated into a vector carrying a neomycin-resistant gene that had been constructed by inserting a promoter of cytomegalovirus and a poly A sequence of SV40 into pSV₂neo (Southern, P. J. and Berg, P., J. Mol. Appl. Genet. 1(4), 327-341(1982)). The expression vector was introduced into K562 cells by means of electroporation method using a gene pulser of Bio-Rad to prepare transformants that express the B7 gene. The transformants were treated with mitomycin or by irradiation to cause no amplification. Further, the transformants were cloned by the limiting dilution-culture method and those having high expression were selected by means of a flowcytometer as K562 cells that express B7 gene, which were used to prepare activated NK cells as described below.

(2) Separation of Lymphocytes

The blood drawn from a patient was poured in a centrifugal tube and diluted to twice with a phosphate buffered solution (PBS), followed by transferring the dilution into lympho-prep tubes. They were centrifuged at 1,500-2,000 rpm at ambient temperature for 15 to 20 minutes, and the resulting lymphocyte layer (a layer so-called as buffy coat) was harvested and transferred into a new centrifugal tube. The buffy coat was washed with about 30-40 ml per tube of PBS or RPMI 1640, and then centrifuged at 1,500 rpm for 10 minutes, to remove the supernatant. This operation was repeated twice. The lymphocytes were adjusted to a predetermined concentration of 4×10 cells per milliliter by adding a human serum-added medium (Hi-Medium930, Kojin Bio, Inc.). The lymphocyte concentration was determined with a blood count plate and the number of lymphocytes was counted as accurately as possible.

(3) Preparation of Activated NK Cells

The lymphocytes as prepared in (2) were stimulated with the K562 cells incorporated with the B7 gene as prepared in (1) every 3-4 days for 3 weeks.

Specifically, to 1−5×10⁶ cells/ml of the lymphocytes in a medium (Hi-Medium930: NIPRO) containing IL-2 and the human serum were added 1/100 to 1/500 amounts of the transformants in the same medium every 3-4 days for 3 weeks, and the mixture was incubated under 5% CO₂ at 37° C.

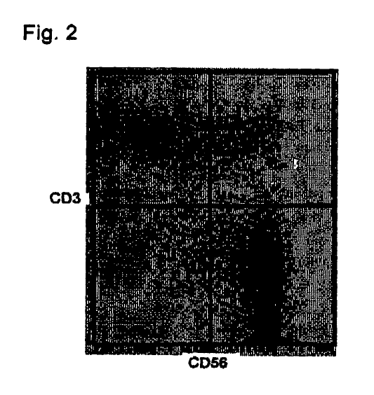

The proliferated (activated) NK cells were assayed for cell surface markers by a FACScan flowcytometry (Beckton-Dickinson) using FITC-labeled anti-CD3 antibody and PE-labeled anti-CD56 antibody. The results are shown in FIG. 2. The horizontal axis indicates the CD56, which represents the amounts of the NK cells. The expression of CD56 is higher as the plot is located to the right side. The vertical axis indicates the CD3, which represents the amounts of T cells. Specifically, the lower right fraction shows the amount of NK cells (70%), whereas the upper left fraction shows the amounts of T cells (18%), suggesting that the procedures as described above produced mainly the activated NK cells of interest.

The result also shows that the stimulation with K562 cells that express B7 gene activated the NK cells in the quantity.

### Example III

Effect of the NK Cells Activated with K562 Cells that Express B7 Gene on the In Vivo Activation of the NK Cells in a Living Body

(1) Administration of Activated NK Cells to a Patient.

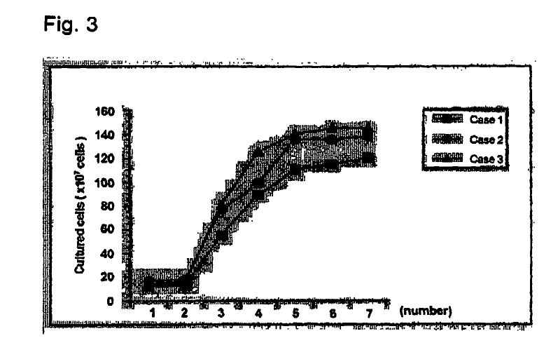

First, 50 ml of a blood sample were drawn from the three patients, and then stimulated as described in Example II for three weeks. The resultant activated NK cells were administered to the same patients in a dose of 100 ml, and, simultaneously, a blood sample was again drawn from the same patients. The resultant blood samples were again stimulated as described in Example II for three weeks. The resultant activated NK cells were administered to the same patients in a dose of 100 ml, and, simultaneously, a blood sample was again drawn from the same patients. This procedure was repeated. The cultured lymphocytes were countered by use of a hemocytometer, and activated NK cells obtained in each stimulation were analyzed by a flowcytometer. The results are shown in FIG. 3. The cultured cells would represent the level of activated NK cells in blood of the patient. FIG. 3 shows that the third administration caused 8 times, and the seventh administration caused 14 times over the first or the second administration, suggesting that the counts of activated NK cells in blood of the patient would not be in proportion to the counts of the administered activated NK cells. This also shows that the administered activated NK cells should activate nonactivated NK cells circulating in a living body.

(2) Examination of Morphology of NK Cells

The fact that activated NK cells that are administered to a patient should activate non-activated NK cells circulating in a living body was confirmed by examining the morphology of NK cells.

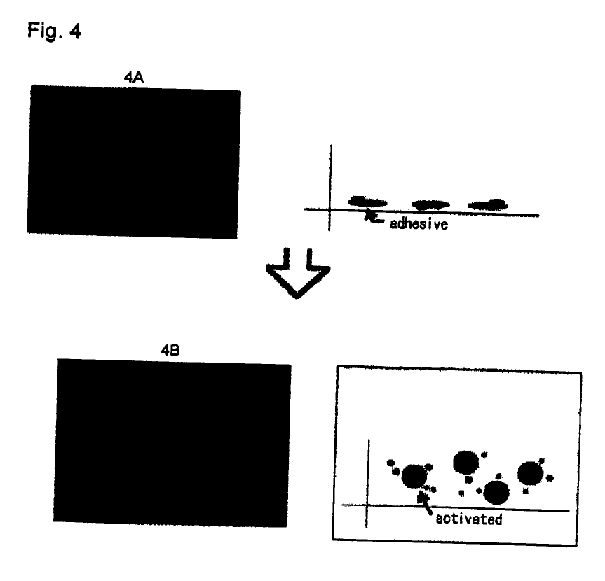

The blood sample was drawn from a cancer patient receiving chemotherapy with anti-cancer agents or radiotherapy. Lymphocytes were isolated from the blood sample, and were incubated in a flask and maintained at a cell density of 5×10⁶/ml. The lymphocytes were observed using an inverted microscope (×40). FIG. 4A shows the photograph and the diagram thereof depicting the morphology of the lymphocytes including NK cells. The lymphocytes adhered to the bottom of the flask, showing that the lymphocytes were poorly proliferated.

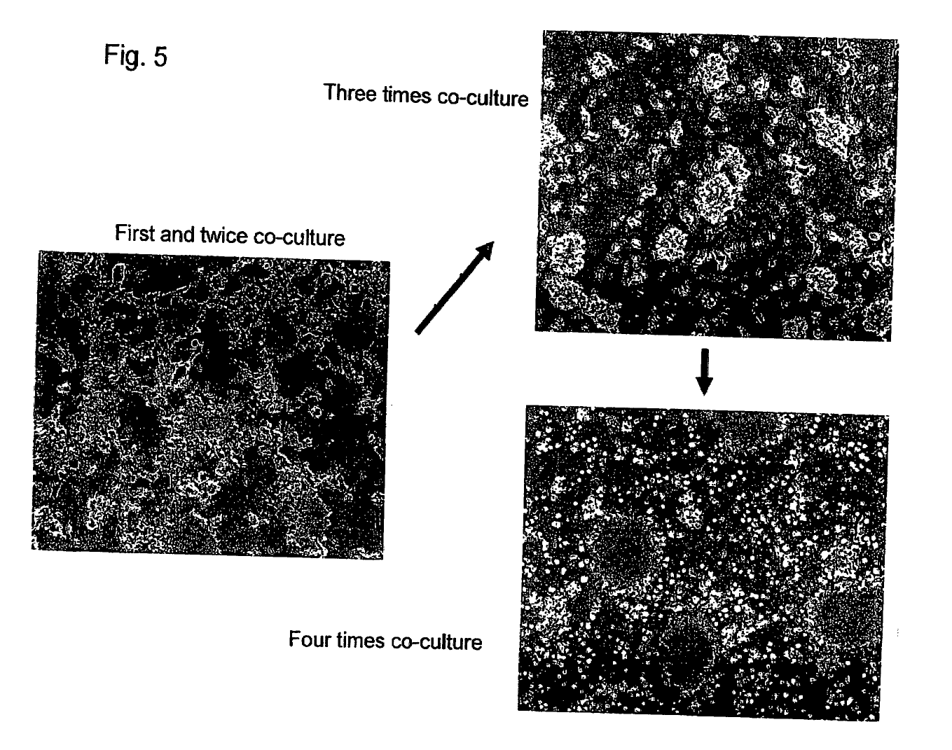

Then, the lymphocytes that were poorly proliferated were stimulated with K562 cells that express B7 gene for two weeks as described in Example II. The resultant activated NK cells were administered to the same patient in a dose of 100 ml, and, simultaneously, a blood sample was again drawn from the same patient. This procedure was repeated four times and more, and, each time, activated NK cells were incubated in a flask and maintained at a cell density of 5×10⁶/ml. The NK cells were observed using an inverted microscope (×40). FIG. 5 shows the photographs depicting the morphology of each NK cells. FIG. 5 demonstrates that NK cells recovered the morphology due to the stimulation with K562 cells that express B7 gene, suggesting the recovery of the activity of NK cells. FIG. 4B also shows that the NK cells stimulated were suspended in a form of the cell mass, showing that the NK cells were proliferated well.

Summing up, the stimulation with K562 cells that express B7 gene was demonstrated to activate the lymphocytes poorly proliferated that circulate in a patient.

### Example IV

Comparison in the Cytotoxic Effects Between Non-Activated NK Cells, T Cells from T-LAK, LAK Cells, and the NK Cells Activated with K562 Cells that Express B7 Gene

(1) Preparation of Various Cells.

In this example, K562 cells and Daudi cells, both of which are the well known cancer cells were used as target cells, and non-activated NK cells, T cells from T-LAK, LAK cells, and the NK cells activated with K562 cells that express B7 gene were used as effector cells.

Peripheral blood lymphocytes (PBL) wherein NK cells account for 10-20% were used as non-activated NK cells. PBLs were prepared by culturing the blood sample in the absence of IL-2 for one day.

The lymphocytes were isolated as described in Example II, and then incubated for two weeks in a flask coated with anti-CD3 antibody to prepare the T cells from T-LAK.

The lymphocytes were incubated for 3-5 days in the presence of a high level of IL2 (1,000 U/ml) to prepare LAK cells.

The activated NK cells were prepared as described in Example II.

(2) Determination of Cytotoxic Effects

Using Delfia system of Wallac, the target cells, K562 cells and Daudi cells, were labeled with europium. Cytotoxic effects of the effector cells were estimated by determining europium that is released into the medium from the lysed cells. Cytotoxicity 100% corresponds to the amount of europium released from 5000 labeled cells/well, all of which were lysed by a detergent.

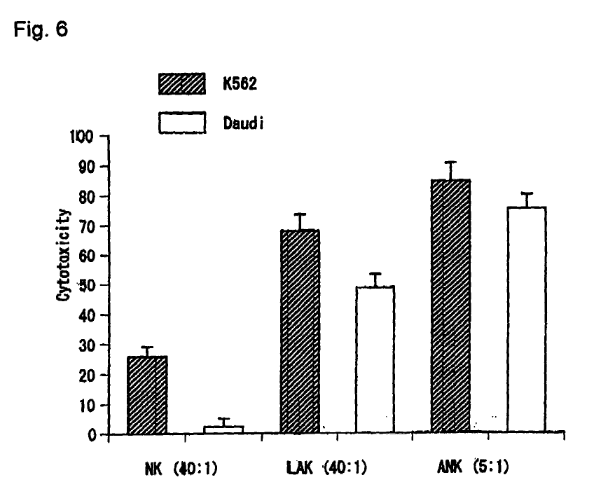

Non-activated NK cells were incubated with the target cells for four hours in an amount of 5 to 40 folds compared to the latter, and then europium released from the target cells was determined and estimated as cytotoxicity %. Similarly, the T cells from LAK and the activated NK cells were used as effector cells, and the europium released from the target cells was estimated as cytotoxicity %. The results are shown in FIG. 6. In FIG. 6, NK means non-activated NK cells, LAK means T cells from LAK, ANK means activated NK cells, and (40:1) means a ratio of Effector cells to Target cells (ET ratio) of 40:1. FIG. 6 shows that non-activated NK cells never kill Daudi cells even in an ET ratio of 40:1.

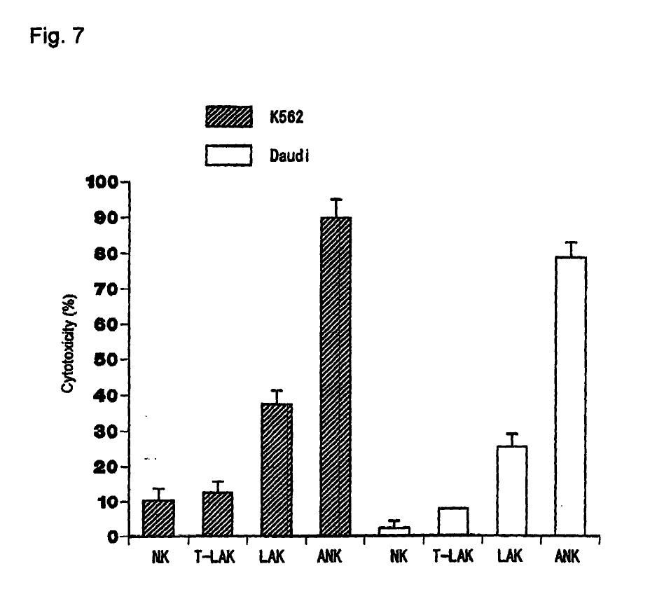

Similarly to the procedure as described above, non-activated NK cells, T cells from T-LAK, LAK cells, and the NK cells activated with K562 cells that express B7 gene were incubated with K562 cells and Daudi cells in a ET ratio of 5:1. The results are shown in FIG. 7. FIG. 7 suggests apparently that the activated NK cells exhibit the strongest cytotoxic effects on the target cells.

### Example V

Effect of K562 Cells that Express B7 Gene on Production of Th1 Cells

The blood sample taken from a patient were contacted to K562 cells as described in Example II so as to obtain a population of the lymphocytes. The population were stimulated for four hours with TPA phorbor ester (12-O-tetradecanolyl-phorbol-13-acetate) and Calcium lonophore (A23187), and then immobilized. After treatment with a detergent that allows antibodies to penetrate into the cell membrane, the immobilized cells of the population were stained with the fluorescence-labeled antibodies against interferon-γ, CD3, CD56, and CD4.

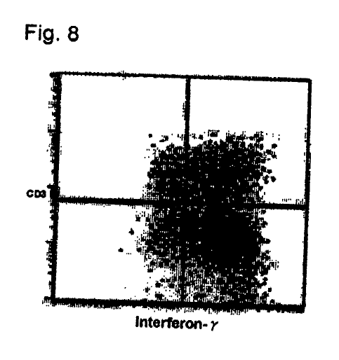

The population was observed by a FACScan flowcytometry (Beckton-Dickinson). The results are shown in FIG. 8. The horizontal axis indicates the level of interferon-γ, which is higher as the plot is located to the right side. The vertical axis indicates the CD3 representing T cells, of which upper from the central axis shows positive whereas the lower shows negative. Specifically, the lower fraction shows the amount of NK cells which produce interferon-γ. The upper fraction shows the amounts of Th1 cells, i.e., T cells that produce interferon-γ. Th1 cells play an important role in cell-mediated immunity, which enhances the immuno-competency against cancers.

The result shows that the stimulation with K562 cells that express B7 gene also activates the production of Th1 cells.

### Example VI

Clinical Trials using the NK Cells Activated with K562 Cells that Express B7 Gene

From the patients, the NK cells activated with K562 cells that express B7 gene were prepared as described in Example II, and then administered to the same patients (hereinafter, referred to as ANK therapy). Prognoses of the patients are described below:

IO: Male, 62 years old; 58 years old when the ANK therapy started; Colon cancer.

He underwent an operation for colon cancer, and his urinary duct was excised due to the development of retroperitoneal infiltration. He was believed to be within one year of death.

ANK therapy of the present invention was conducted twice a week during the period of the first year, once a week during the period of the following six months, once every two weeks during the period of the following six months, and then once a month.

Diagnosis with tumor markers and imaging revealed that there is still no relapse.

HM: Male; died at 78 years old; 71 years old when the ANK therapy started; Hepatocellular carcinoma. Metastasis into lung.

ANK therapy of the present invention was conducted three times a week during the period of the first six months, twice a week during the period of the following six months, and then once a month.

Both cancers disappeared. He was dead from cerebral infarction.

MK: Male; 52 years old when the ANK therapy started; Leiomyosarcoma.

Hepatic metastasis of the sarcoma and the strong jaundice were found. He was believed to be within one month of death.

ANK therapy of the present invention was conducted twice a week during the period of the first six months, once a week during the period of the following three months, and then once a month. The cancer was drastically decreased, and almost disappeared after one year. The ANK therapy was terminated by his intention although he was recommended to continue the ANK therapy for treatment of the remaining affected area. He lived three years after the termination of the ANK therapy until dead of the relapse.

MH: Male, 65 years old; 58 years old when the ANK therapy started; Malignant lymphoma.

He was believed to be within one year of death. ANK therapy of the present invention was conducted twice a week. The cancer drastically disappeared, and he went into remission. He has been still alive even seven years after the starting of the therapy.

MH: Male, 53 years old; 50 years old when the ANK therapy started; Nasopharyngeal carcinoma.

The carcinoma was hardly excised, and his prognosis was believed worse. ANK therapy of the present invention was conducted twice a week during the period of the six months, and then once a week during the six months. There is no relapse still at the present.

MI: Female, 48 years old; 45 years old when the ANK therapy started; Relapse of mammary cancer, Two metastases into lung.

Chemotherapy did not improve the cancer. ANK therapy of the present invention using lymphocytes took before the chemotherapy was conducted twice a week during the first six months, and then one of the cancers disappeared whereas the other was reduced in size. At the present, she receives Picibanil. She has been into remission.

SS: Male, 74 years old; 73 years old when the ANK therapy started; Prostatic cancer, Multiple metastases into lung.

ANK therapy of the present invention was conducted twice a week during the period of the first six months, and then once a month. Almost all cancers but one cancer disappeared. At the present, the ANK therapy was continued.

HU: Male, 61 years old; 58 years old when the ANK therapy started; Malignant lymphoma.

ANK therapy of the present invention was conducted twice a week for one year. There is no relapse still at the present.

MH: Male, 73 years old; 67 years old when the ANK therapy started; Prostatic cancer.

ANK therapy of the present invention was conducted twice a week for six months. The high PSA was reduced to the normal level. There is no relapse still at the present.

IO: Male, Died at 72 years old (due to myocardial infarction); 69 years old when the ANK therapy started; Prostatic cancer.

ANK therapy of the present invention was conducted twice a week for six months. The high PSA was reduced to the normal level. There was no relapse, until the patient died of myocardial infarction.

TK: Male, 69 years old; 63 years old when the ANK therapy started; Nasopharyngeal carcinoma.

ANK therapy of the present invention was conducted twice a week during the period of one month, and then the diagnosis revealed that the carcinoma disappeared. The ANK therapy has been still continued twice a week during the period of six months.

HI: Male, 69 years old; 65 yeas old when the ANK therapy started; Nasopharyngeal carcinoma.

ANK therapy of the present invention was conducted twice a week during the period of six months, and then the carcinoma was reduced in size. Although the ANK therapy has not been continued, he is still alive.

JN: 75 years old; 71 yeas old when the ANK therapy started; Inoperable pancreatic adenocarcinoma.

ANK therapy of the present invention was conducted once every three weeks, and then the progression was terminated. The patient is still alive.

TN: 71 yeas old; 68 years old when the ANK therapy started; Inoperable pancreatic adenocarcinoma.

ANK therapy of the present invention was conducted twice a week during the period of six months. The progression of the disease was terminated. The patient was released from pain, and could take some meals. The patient is still alive.

### EFFECTS OF THE INVENTION

As is described above, the method of the in vitro culture of lymphocytes can amplify a group of lymphocytes composed mainly of NK cells and/or killer T cells specific to cancer in a high yield and in a stable manner by incubating a mixture of lymphocytes with cells at a predetermined rate, in which a particular expression gene such as B7 gene or a cancer antigen gene has been introduced in particular cancer cells and the particular expression gene has been expressed or in which such a particular expression gene has already been expressed. The lymphocytes group presents the extremely great merits that it can be used as a source of an effective immune treatment even for cancer patients to which conventional cancer therapy has been found ineffective, so that the present invention can realize a remarkably effective cancer treatment.

The group of the lymphocytes prepared by the in vitro culture according to the present invention can provide the great merits that it can be used for the immune treatment for cancer as an reagent for immunologically treating cancer, which does not involve oncogenesis, in place of the conventional method that uses B cells mutated by virus involved with oncogenesis.

Further, the method for the in vitro culture of the lymphocytes according to the present invention can selectively induce and amplify killer cells from the lymphocytes having killer activity, which adapt to the individual patient. Therefore, particularly through effective destruction of cancer cells, the killer T cells specific to the cancer antigen can be also amplified in a stable way so that the present invention can be applied as a source of an effective immune therapy even for the patients with cancer, to which the conventional cancer therapy has been ineffective, so that the present invention can provide the great merits that it can realize a remarkably effective cancer treatment.

1. A process for preparing activated NK cells in vitro, which comprises the steps of:
a) isolating lymphocytes from a subject; and
b) incubating the lymphocytes together with a cancer cell which expresses an immunoglobulin superfamily gene, thereby activating NK cells included in the isolated lymphocytes,
wherein the cancer cell is deficient or decreased in the expression of a class I antigen, and wherein the immunoglobulin superfamily gene encodes a cell adhesion molecule.

2. The process of claim 1, wherein the cancer cell is selected from a group consisting of K562 cells and Daudi cells, and the immunoglobulin superfamily gene is selected from a group consisting of B7 gene, CD40, LFA-1, and a combination thereof.

3. A process for activating NK cells that circulates in a subject, which comprises administering the activated NK cells as prepared according to the process of claim 1 or 2 to the subject.

4. A process for immunostimulation in a cancer patient, which comprises the steps of:
a) isolating lymphocytes from the patient;
b) incubating the lymphocytes together with a cancer cell which expresses an immunoglobulin superfamily gene, thereby activating NK cells included in the isolated lymphocytes; and
c) administering the activated NK cells to the same cancer patient,
wherein the cancer cell is deficient or decreased in the expression of a class 1 antigen, and wherein the immunoglobulin superfamily gene encodes a cell adhesion molecule.

5. The process of claim 4, wherein the cancer cell is selected from a group consisting of K562 cells and Daudi cells, and the immunoglobulin superfamily gene is selected from a group consisting of B7 gene, CD40, LFA-1, and a combination thereof.

6. The process for immunostimulation of claim 4 or 5, wherein the steps thereof are repeated three times or more.

7. A method for treating a cancer disease in a patient, which comprises the steps of:
a) isolating lymphocytes from the patient;
b) incubating the lymphocytes together with a cancer cell which expresses an immunoglobulin superfamily gene, thereby activating NK cells included in the isolated lymphocytes; and
c) administering the activated NK cells to the same cancer patient,
wherein the cancer cell is deficient or decreased in the expression of a class I antigen, and wherein the immunoglobulin superfamily gene encodes a cell adhesion molecule.

8. The method of claim 7, wherein the cancer cell is selected from a group consisting of K562 cells and Daudi cells, and the immunoglobulin superfamily gene is selected from a group consisting of B7 gene, CD40, LFA-1, and a combination thereof.

9. The method for treating a cancer disease according to claim 7 or 8, wherein the steps thereof are repeated three times or more.

10. A process for inducing the proliferation of activated NK cells in vitro, which comprises:
a) isolating lymphocytes from a subject; and
b) incubating the lymphocytes together with a cancer cell which expresses an immunoglobulin superfamily gene, thereby activating NK cells included in the isolated lymphocytes,
wherein the cancer cell is deficient or decreased in the expression of a class I antigen, and wherein the immunoglobulin superfamily gene encodes a cell adhesion molecule.

11. The process of claim 10, wherein the cancer cell is selected from a group consisting of K562 cells and Daudi cells, and the immunoglobulin superfamily gene is selected from a group consisting of B7 gene, CD40, LFA-1, and a combination thereof.

12. The process of claim 10 or 11, wherein the proliferation of Th1 cells are further induced.

13. The process of claim 10 or 11, wherein the subject is a healthy subject, and the activated NK cells are reserved in preparation for possible cancer diseases.

14. A method for treating a cancer disease in a patient, which comprises administering an aliquot of the reserved activated NK cells according to claim 13 to the patient, wherein the reserved activated NK cells were obtained from the patient when the patient was previously healthy.
